(19) United States
(12) Patent Application Publication
(10) Pub. No.: US 2024/0412356 A1
(43) Pub. Date: Dec. 12, 2024

(54) SYSTEM AND METHOD OF CELL ANOMALY DETECTION

(71) Applicant: B.G. Negev Technologies & Applications Ltd., at Ben-Gurion University, Beer-Sheva (IL)

(72) Inventors: Assaf ZARITSKY, Beer-Sheva (IL); Naor KOLET, Beer-Sheva (IL)

(73) Assignee: B.G. Negev Technologies & Applications Ltd., at Ben-Gurion University, Beer-Sheva (IL)

(21) Appl. No.: 18/699,778

(22) PCT Filed: Oct. 30, 2022
(86) PCT No.: PCT/IL2022/051151

§ 371 Date: Apr. 9, 2024

Related U.S. Application Data
(60) Provisional application No. 63/272,691, filed on Oct. 28, 2021.

(51) Int. Cl. G06T 7/00; G16H 20/10; G16H 50/20

(52) U.S. Cl. CPC G06T 7/0012; G16H 20/10; G16H 50/20; G06T 2207/10056; G06T 2207/20084

(57) ABSTRACT

The present invention relates generally to machine learning and artificial intelligence methods of cell anomaly detection. More specifically, the present invention relates to targeting intracellular anomalies via microscopy-based high-content phenotypic screening and generative neural networks for combinatorial drug screening. As can be seen from the provided description, the claimed invention represents the system and method of cell anomaly detection, which increase reliability of cell anomaly detection. More specifically, the claimed invention provides an assessment of cell inter-component (inter-organelle) organization for detecting cell anomaly.

### CROSS-REFERENCE TO RELATED APPLICATIONS

This application claims the benefit of priority of U.S. Patent Application No. 63/272,691, filed Oct. 28, 2021, and entitled: “Targeting intracellular organization via microscopy-based high-content phenotypic screening and generative neural networks for combinatorial drug screening” which are all hereby incorporated by reference in their entirety.

### FIELD OF THE INVENTION

The present invention relates generally to machine learning and artificial intelligence methods of cell anomaly detection. More specifically, the present invention relates to targeting intracellular anomalies via microscopy-based high-content phenotypic screening and generative neural networks for combinatorial drug screening.

### BACKGROUND OF THE INVENTION

Cellular organelle and subcellular organization form the underlying basis for cellular complexity. While many studies known form the art focus on the role and structure of individual organelles, the major subcellular structures that compose a cell, much less is known about how different organelles coordinate their organization and function within the cell, a fundamental requirement for the observed integrated complex cell function. As known from the art, dysfunctions in inter-organelle organization were recently linked to several diseases, for example, defects in membrane contact sites between the endoplasmic reticulum (ER) and the mitochondria are thought to have implications in cancer, neurodegenerative disorders and diabetes. More specifically, ER and mitochondrial dysfunction and structural changes in Idiopathic pulmonary fibrosis, organelle acidification and ER stress in cystic fibrosis. Dissociation of the ER from the mitochondria is found in some cases of type 2 diabetes that could be related to increased insulin resistance. Chronic hypoxia and acute kidney injury shown decreased mitochondrial activity which also affects ER activity.

As known, visual phenotypes in cell appearance are determined by the cell organelles composition in space and proper organelle-organelle organization and provide clues for proper or impaired cell function in health and disease. High-content image-based cell phenotyping using automated microscopy is emerging as a power data-driven approach to identify differences in cell populations with applications in identifying phenotypes signatures of specific diseases, drug screening, clustering compounds/genes into functional pathways, and identifying drugs targets/mechanisms of action. For example, image-based phenotyping can be a very effective strategy to identify potential molecular targets or for repurposing approved drugs. The potential benefit of this approach stems from its ability to evaluate the potential efficacy and safety of thousands to millions of drugs at the single cell resolution to significantly relieve a key bottleneck at the initial stages of the drug discovery process prior to treating patients with candidate drugs. Accordingly, major pharma companies as well as startups companies are implementing high-content image-based cell phenotyping techniques as part of their R&D efforts.

The “Cell Painting” assay, where each image is composed of five fluorescent channels marking different cell organelles, was recently developed for high-content phenotyping, and provides a rich morphological cell descriptor suitable to identify subtle phenotypes with high sensitivity. As known, cell painting is becoming the assay of choice for phenotypic screening in academia and in the pharma industry. In fact, a recent joint efforts of major pharma companies and academia are focused on generating public data sets at unprecedented scale to enable validations and scaling up Cell Painting-based image-based drug discovery strategies.

### SUMMARY OF THE INVENTION

Accordingly, there is a need for a system and method of cell anomaly detection, which would increase reliability of cell anomaly detection. More specifically, there is a need to provide an assessment of cell inter-component (inter-organelle) organization for detecting cell anomaly.

To overcome the shortcomings of the prior art, the following invention is provided.

In general aspect, the invention may be directed to a method of cell anomaly detection by at least one processor, the method including receiving a set of cell component data elements in an original version, wherein each cell component data element represents a distinct cell component type of a cell; inferring at least one pretrained machine learning (ML)-based model on at least one first cell component data element of the set of cell component data elements in the original version, to obtain at least one second cell component data element of the set of cell component data elements in a reconstructed version; classifying the cell as having an anomaly based on the reconstructed version of at least one second cell component data element.

In another general aspect, the invention may be directed to a method of cell anomaly detection by at least one processor, the method including receiving a plurality of sets of cell component data elements in an original version, wherein each set corresponds to a distinct cell and each cell component data element within each set represents a distinct cell component type of a respective cell; forming a training dataset, including examples of mapping between at least one first cell component data element of at least one first set of the plurality of sets of cell component data elements in the original version and at least one second cell component data element of the at least one first set in the original version; by using the training dataset, training at least one machine learning (ML)-based model to reconstruct, based on the at least one first cell component data element of the at least one first set in the original version, at least one second cell component data element of the at least one first set in the original version, and obtain thereby the at least one second cell component data element of the at least one first set in a reconstructed version; inferring the pretrained at least one ML-based model on at least one first cell component data element of a second set of the plurality of sets of cell component data elements in the original version, to obtain at least one second cell component data element of the second set in the reconstructed version; classifying the respective cell as having an anomaly based on the reconstructed version of the at least one second cell component data element of the second set.

In yet another general aspect, the invention may be directed to a system for cell anomaly detection, the system including a non-transitory memory device, wherein modules of instruction code are stored, and at least one processor associated with the memory device, and configured to execute the modules of instruction code, whereupon execution of said modules of instruction code, the at least one processor is configured to receive a set of cell component data elements in an original version, wherein each cell component data element represents a distinct cell component type of a cell; infer at least one pretrained machine learning (ML)-based model on at least one first cell component data element of the set of cell component data elements in the original version, to obtain at least one second cell component data element of the set of cell component data elements in a reconstructed version; and classify the cell as having an anomaly based on the reconstructed version of at least one second cell component data element.

In some embodiments, classifying the cell as having an anomaly includes calculating a reconstruction error value based on the original version and the reconstructed version of the at least one second cell component data element; and classifying the cell as having an anomaly, further based on the calculated reconstruction error value.

In some embodiments, classifying the cell as having an anomaly based on the calculated reconstruction error value further includes classifying the cell as having an anomaly by determining that the calculated reconstruction error value is higher than a predefined reconstruction error threshold value.

In some embodiments, the at least one pretrained ML-based model is pretrained so as to produce the at least one second cell component data element in the reconstructed version, based on the at least one first cell component data element in the original version.

In some embodiments, the at least one cell component data element is a microscopy image of the cell.

In some alternative embodiments, the at least one original data element is a vector representation of a set of features extracted from a microscopy image of the cell.

In some embodiments, the set of cell component data elements includes n distinct combinations of the at least one first and at least one second cell component data elements; and the at least one ML-based model comprises n ML-based models, each corresponding to a respective combination of the n distinct combinations.

In some embodiments, each ML-based model of the n ML-based models is pretrained to obtain the at least one second cell component data element in the reconstructed version, based on the at least one first cell component data element in the original version, according to the respective combination of the n distinct combinations.

In some embodiments, inferring the at least one pretrained machine learning (ML)-based model includes inferring each ML-based model of the n ML-based models on the at least one first cell component data element of the respective combination in the original version, to obtain the at least one second cell component data element of the respective combination in the reconstructed version.

In some embodiments, classifying the cell as having an anomaly includes, for each combination of the n distinct combinations, calculating a reconstruction error value based on the original version and the reconstructed version of at least one respective second cell component data element; classifying the cell as having an anomaly, further based on the calculated reconstruction error values.

In some embodiments, classifying the cell as having an anomaly based on the calculated reconstruction error values further includes classifying the cell as having an anomaly by determining that at least one of the calculated reconstruction error values is higher than a respective predefined reconstruction error threshold value.

In some embodiments, the method further includes calculating a first reconstruction error value based on the original version and the reconstructed version of the at least one second cell component data element of the at least one first set; and defining a reconstruction error threshold value based on the first reconstruction error value.

In some embodiments, the at least one first set includes a plurality of the first sets; and defining a reconstruction error threshold value based on the first reconstruction error value includes defining a reconstruction error threshold value based on the distribution of first reconstruction error values within the plurality of first sets.

In some embodiments, classifying the respective cell as having an anomaly includes calculating a second reconstruction error value based on the original version and the reconstructed version of the at least one second cell component data element of the at least one second set; classifying the respective cell as having an anomaly by determining that the second reconstruction error value is higher than the predefined reconstruction error threshold value.

In some embodiments, the at least one first set of the plurality of sets of cell component data elements in the original version corresponds to a distinct control cell of a cell-based research and the at least one second set of the plurality of sets of cell component data elements in the original version corresponds to a distinct perturbed cell of the cell-based research.

In some embodiments, the at least one ML-based model is a generative deep neural network.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 represents microscopy images, depicting an example of Cell Painting assay;

FIG. 2 is a schematic representation of the hypothesis underlying the claimed invention;

FIG. 3 is a schematic representation of the concept of the claimed invention;

FIG. 4 is a block diagram, depicting a computing device which may be included in the system for training ML-based model, according to some embodiments;

FIG. 5 is a general representation of a concept applied by embodiments of the claimed invention;

FIG. 6 is a block diagram, depicting a system for cell anomaly detection, according to some embodiments;

FIG. 7 is a plot, explaining evaluation of the magnitude of perturbed inter-organelle organization vs. the corresponding organelle properties;

FIG. 8 is a set of plots, depicting normalized deviation in the corresponding organelle properties in relation to the deviation in the inter-organelle organization;

FIG. 9 is a set of plots, depicting replication of anomaly detection results across different plates during approbation of the claimed invention.

FIG. 10 is a set of plots, depicting sensitivity and specificity of inter-organelle organization approach in relation to the single organelle approach;

FIG. 11 is a schematic representation, depicting anomaly detection for ML-based model explainability;

FIG. 12A is a flow diagram, depicting a method of cell anomaly detection, according to some embodiments;

FIG. 12B is a flow diagram, depicting a method of cell anomaly detection, according to other embodiments.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

The terms “intracellular”, “inter-organelle”, “inter-component”, “organelle-organelle” may be used herein interchangeably and refer to organization and interconnection between components within a cell.

The terms “anomaly” and “alteration” may be used herein interchangeably and refer to changes in inter-component organization within a cell in relation to normal organization.

The terms “organelle” and “component” may be used herein interchangeably and refer to a content of a cell.

The terms “organization”, “special dependency”, “composition”, “appearance”, “structure”, “molecular organization” and “phenotype” may be used herein interchangeably and refer to the arrangement of and relations between the components, parts or elements of a cell.

The terms “channel”, “modality”, “imaging modality” may be used herein interchangeably and refer to a representation of a distinct cell component type (organelle type) of a cell.

The terms “data element”, “image”, “set of features” may be used herein interchangeably and refer to specific way of representation of cell and cell components.

The terms “model”, “ML-based model”, “autoencoder”, “network”, “neural network”, “generative adversarial network” may be used herein interchangeably and refer to machine-learning model used in claimed method or system.

The terms “control cell” and “control” may be used herein interchangeably and refer to control cells of a cell-based research.

The terms “perturbed cell” and “perturbation” may be used herein interchangeably and refer to perturbed cells of a cell-based research.

The terms “predict”, “reconstruct”, “obtain”, “calculate” may be used herein interchangeably and refer to the processing of an input data by ML-based model.

It should be understood that, in the context of the application, terms used herein interchangeably may be marked and referred by same numerical identifiers.

Although embodiments of the invention are not limited in this regard, discussions utilizing terms such as, for example, “processing,” “computing,” “calculating,” “determining,” “establishing”, “analyzing”, “checking”, “choosing”, “selecting”, “omitting”, “training” or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, concurrently, or iteratively and repeatedly.

In some embodiments of the present invention, ML-based model may be an artificial neural network (ANN).

A neural network (NN) or an artificial neural network (ANN), e.g., a neural network implementing a machine learning (ML) or artificial intelligence (AI) function, may refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. A processor, e.g., CPUs or graphics processing units (GPUs), or a dedicated hardware device may perform the relevant calculations.

It should be obvious for the one ordinarily skilled in the art that various ML-based models can be implemented without departing from the essence of the present invention. It should also be understood, that in some embodiments ML-based model may be a single ML-based model or a set (ensemble) of ML-based models realizing as a whole the same function as a single one. Hence, in view of the scope of the present invention, the abovementioned variants should be considered equivalent.

As can be seen, the concept underlying the claimed invention is based on using one cell component data element (e.g., microscopy image of a cell component) to reconstruct the other cell component data element, wherein each cell component data element represents a distinct cell component type, based on statistical methods. It should be understood that such a concept is thus inherently based on the assessment of cell inter-component (inter-organelle) organization. Statistical data is used to define normal cell inter-component organization and, consequently, to form a ground for anomaly detection in cell single-component/inter-component organization. Therefore, reliability of cell anomaly detection may be dramatically increased.

It should be understood that the claimed invention is not limited by specific techniques, providing input data for the claimed system, e.g., Cell painting assay. Indication of specific techniques herein should be considered as an illustrative non-exclusive example, and other known techniques may be implemented not being out of the scope of the claimed invention. Such techniques may include, for example, spatial omics multiplexing, such as MIBI-TOF.

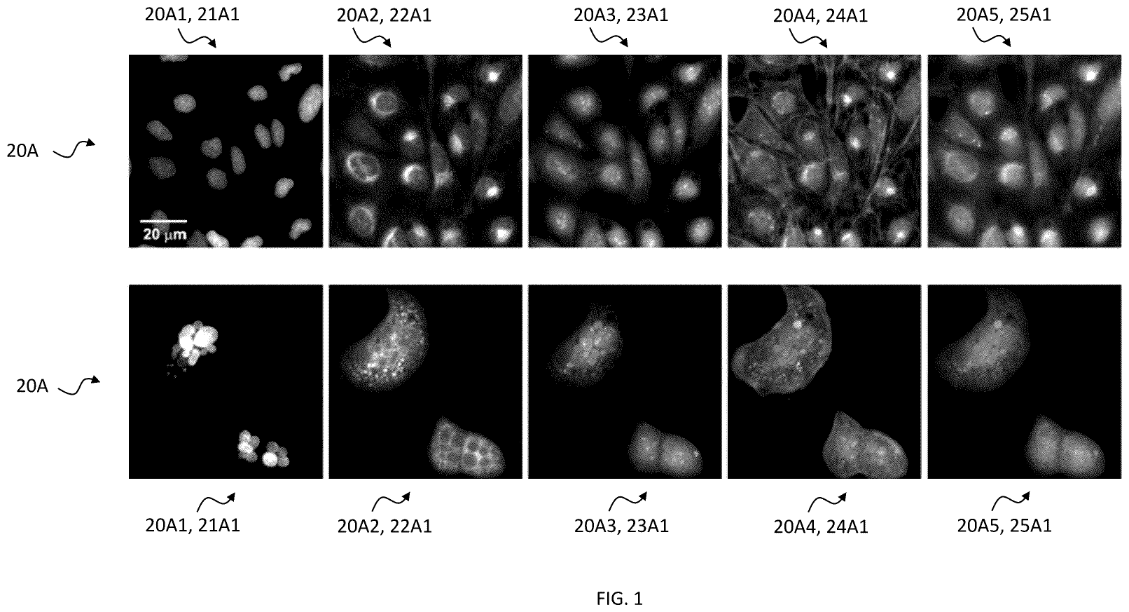

Reference is now made to FIG. 1, which represents microscopy images, depicting an example of Cell Painting assay.

The illustrated Cell Painting assay is of U2OS cells. Five channels (single-channel data elements 20A1, 20A2, 20A3, 20A4 and 20A5) imaged in a DMSO (unperturbed, top row) and a parbendazole (bottom row) well (representing two sets 20A of single-channel data elements): Hoechst 33342 (DNA), concanavalin A (ER), SYTO 14 (nucleoli and cytoplasmic RNA), phalloidin (actin) and WGA (Golgi and plasma membrane), and MitoTracker Deep Red (mitochondria) (referred herein as cell component types 21A1, 22A1, 23A1, 24A1 and 25A1, respectively). Scale bars, 20 μm.

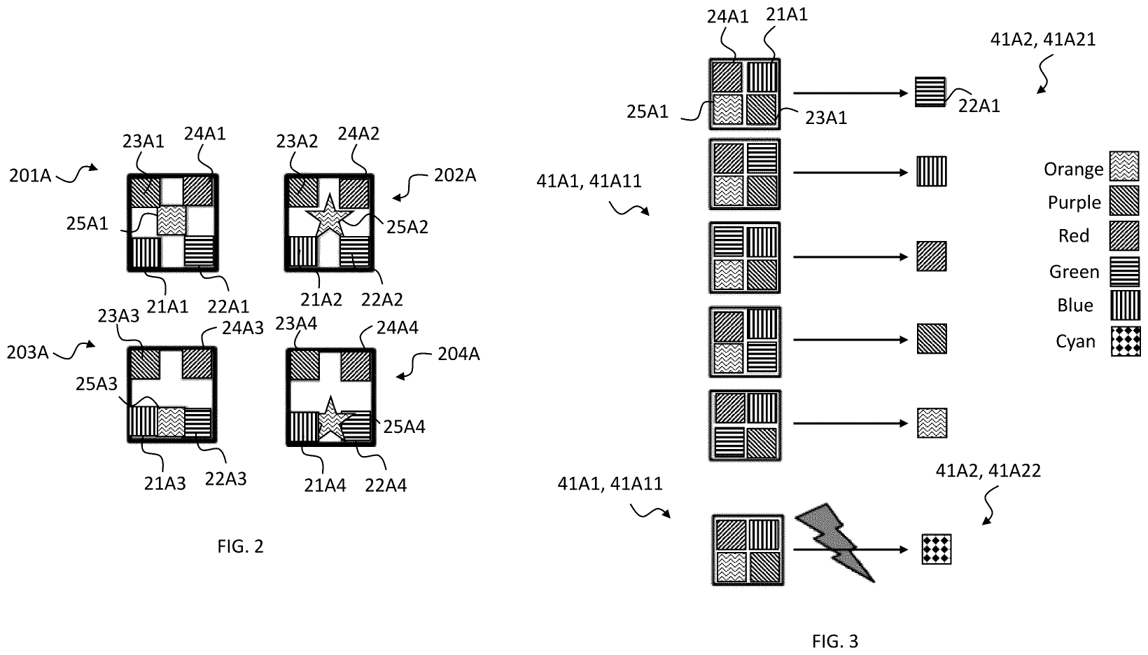

Reference is now made to FIG. 2, which is a schematic representation of the hypothesis underlying the claimed invention.

Hypothesis: spatial inter-organelle organization provide a complementary and sensitive readout for cell state. Schematic representation of the spatial organization includes five organelles (color code), referred herein as cell component types 21A1, 22A1, 23A1, 24A1 and 25A1, respectively, in Control cell 201A (upper left). Current readouts are designed for identifying alterations in single organelles, referred herein as cell component types 21A2, 22A2, 23A2, 24A2 and 25A2, respectively, in cell 202A. Upper right (cell 202A): orange star represents an anomaly in cell component type 25A2. The proposed readout is designed to identify alterations in organelle-organelle (inter-organelle, inter-component) spatial dependencies (organization), e.g., organization between cell component types 21A3, 22A3, 23A3, 24A3 and 25A3, as shown in cell 203A. As shown in bottom left corner: orange square changing its position in relation to the other squares. Proposed readout is expected to capture alterations (anomalies) in inter-organelle organization that are not detected with the current readouts (as indicated in bottom left corner with respect to cell 203A), and to be more sensitive when both organelle and inter-organelle organizations are altered (as indicated in bottom-right corner with respect to cell 204A, including cell component types 21A4, 22A4, 23A4, 24A4 and 25A4).

Thus, while current computational approaches extract and pool image-based features from all Cell Painting fluorescent channels (e.g., single-channel data elements 20A1, 20A2, 20A3, 20A4 and 20A5), the claimed invention proposes to derive specific measures to capture the spatial dependencies between different organelles (e.g., cell component types 21A3, 22A3, 23A3, 24A3 and 25A3) and cellular structures as a new functional readout for spatial inter-organelle organization with applications in image-based phenotyping. The novelty and potential impact of proposed solution stems from the computational definition of quantitative measures designed to capture perturbation-induced alterations in the spatial inter-organelle organization. Each measure may encode a fluorescent channel-specific alteration, dependent on the mapping from the other four channels. These measures will enable mechanistic interpretability of the effects each treatment has on specific aspects of cell organization in terms of “breaking” existing relations between multiple cell structures.

Reference is now made to FIG. 3, which is a schematic representation of the concept of the claimed invention.

As can be seen, cell component data elements, representing cell component types 21A1, 22A1, 23A1, 24A1 and 25A1, respectively, are combined into respective number of combinations. Each combination includes first cell component data elements 41A1 in original version 41A11 and second cell component data element 41A2 in original version 41A21.

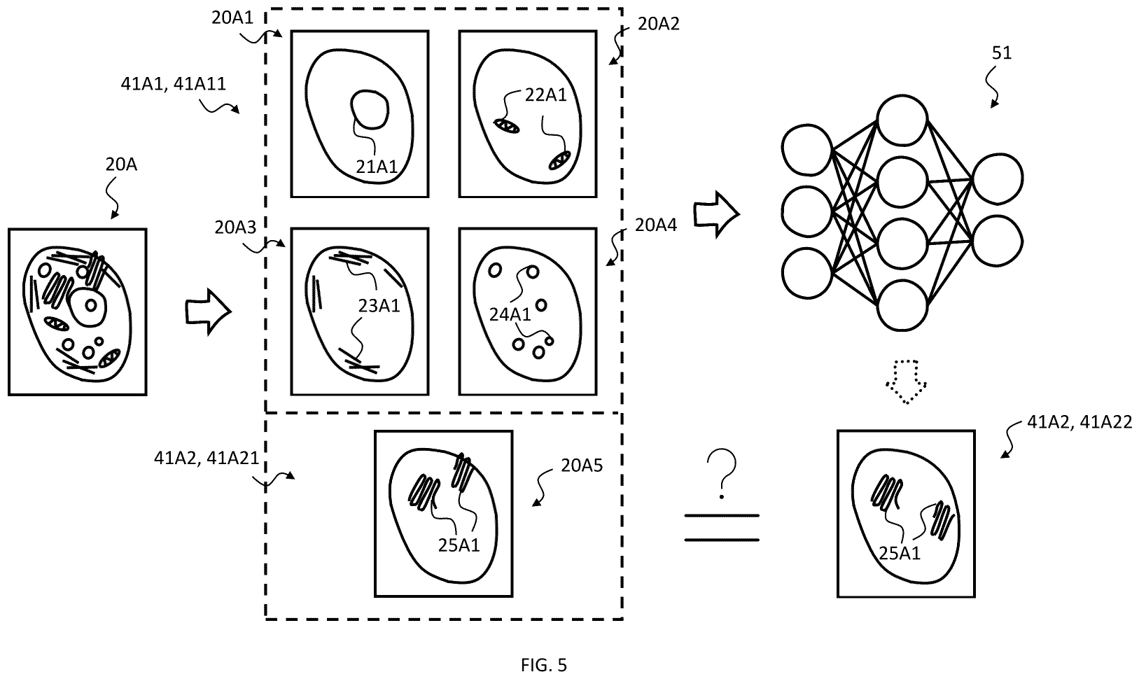

The claimed approach has the following schematics: the first stage (A) is training of models (ML-based models) to map organelle-specific spatial dependency on the other organelles in control cells (based on examples of mapping between first and second cell component data elements 41A1 and 41A2 in their original versions 41A11 and 41A21 respectively, as further described in detail herein with reference to FIG. 5); at the second stage (B) each trained model (ML-based model) may define a mechanistic interpretable measure for the alteration of a specific organelle spatial dependency that was induced by a perturbation (i.e., based on first cell component data elements 41A1 in original version 41A11 of a respective combination, reconstruct second cell component data element 41A2 and obtain thereby the second cell component data element 41A2 in reconstructed version 41A22, with respect to a perturbed cell).

The feasibility of this approach stems from recent studies, known from the art, that applied deep learning based generative models to establish the existence of unstructured hidden information that can be used to map one microscopy image modality to predict the molecular organization of another imaging modality within the cell. During preliminary tests of practical implementation of the invention, it was verified that single cell features extracted from one channel can be accurately predicted from engineered features extracted from the remaining four channels (e.g., single-channel data elements 20A1, 20A2, 20A3, 20A4 and 20A5). Preliminary results (further described in detail) indicate that the proposed measurements for inter-organelle organization provide three important advantages over existing readouts: (1) complementary phenotypic information that is currently missed, (2) a more sensitive readout, (3) a more specific and interpretable readout.

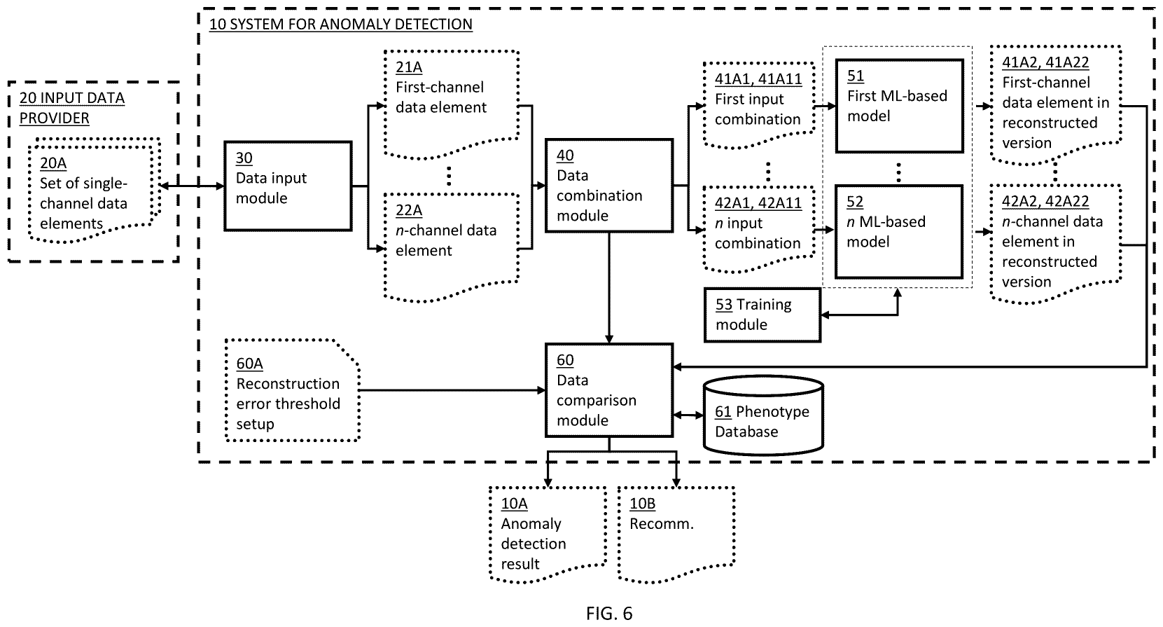

In respect to the illustrated example, to map different combinations of inter-organelle dependencies in control cells, five generative deep neural networks may be trained, each using a different combination of 4-to-1 channels (e.g., combinations of first and second cell component data elements 41A1 and 41A2) of the five fluorescent image channels (e.g., single-channel data elements 20A1, 20A2, 20A3, 20A4 and 20A5). Importantly, when applied to specific cell-based research, each high-content screen contains many replicates of the unperturbed condition providing sufficient data to train such models. Next, measures of similarity in the space defined by the five “control model” networks, between the mapped and the ground truth images, may be defined and used further to quantify and map the alteration following a perturbation in relation to the model trained using control data (i.e., the reconstruction error values may be calculated, and reconstruction error threshold value may be defined respectively, as further described in detail with reference to FIGS. 5 and 6). An important technical detail is the assessment of batch effects, namely intra-well, inter-well, and inter plate variability in unperturbed conditions, which is a confounding factor of getting reliable results.

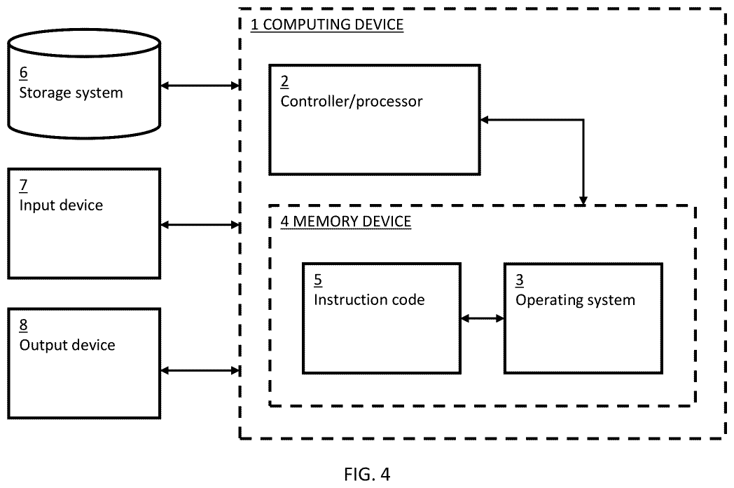

Reference is now made to FIG. 4, which is a block diagram depicting a computing device, which may be included within an embodiment of the system for cell anomaly detection, according to some embodiments.

Computing device 1 may include a processor or controller 2 that may be, for example, a central processing unit (CPU) processor, a chip or any suitable computing or computational device, an operating system 3, a memory device 4, instruction code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to instruction code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory device 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short-term memory unit, a long-term memory unit, or other suitable memory units or storage units. Memory device 4 may be or may include a plurality of possibly different memory units. Memory device 4 may be a computer or processor non-transitory readable medium, or a computer non-transitory storage medium, e.g., a RAM. In one embodiment, a non-transitory storage medium such as memory device 4, a hard disk drive, another storage device, etc. may store instructions or code which when executed by a processor may cause the processor to carry out methods as described herein.

Instruction code 5 may be any executable code, e.g., an application, a program, a process, task, or script. Instruction code 5 may be executed by processor or controller 2 possibly under control of operating system 3. For example, instruction code 5 may be a standalone application or an API module that may be configured to perform cell anomaly detection as further described herein. Although, for the sake of clarity, a single item of instruction code 5 is shown in FIG. 1, a system according to some embodiments of the invention may include a plurality of executable code segments or modules similar to instruction code 5 that may be loaded into memory device 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Various types of input and output data may be stored in storage system 6 and may be loaded from storage system 6 into memory device 4 where it may be processed by processor or controller 2. In some embodiments, some of the components shown in FIG. 1 may be omitted. For example, memory device 4 may be a non-volatile memory having the storage capacity of storage system 6. Accordingly, although shown as a separate component, storage system 6 may be embedded or included in memory device 4.

Input devices 7 may be or may include any suitable input devices, components, or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output devices 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

Reference is now made to FIG. 5, which depicts a general representation of the concept applied by embodiments of the claimed invention.

As described above, the concept is based on using set of cell component data elements represented in original version (e.g., set 20A of single-channel data elements 20A1, 20A2, 20A3, 20A4 and 20A5) for ML-based cell anomaly detection. Each cell component data element may represent a distinct cell component type of a cell (e.g., cell component types 21A1, 22A1, 23A1, 24A1 and 25A1).

In some embodiments, cell component data elements may be microscopy images of a cell, e.g., produced based on known high-content image-based cell phenotyping techniques. In alternative embodiments, cell component data elements may be vector representations of sets of features extracted from microscopy images of a cell. In yet another alternative embodiments, cell component data elements may be images obtained by “spatial” techniques such as (spatial) mass spectrometry. In yet another alternative embodiments, cell component data elements may be images obtained by multiplexing techniques such as spatial omics (transcriptomics, proteomics).

Cell component types may refer to nucleus, endoplasmic reticulum, nucleoli, cytoplasmic RNA etc. It should be understood that the concept is not limited by specific cell components and their combinations. All mentioned cell components are represented herein as an illustrative non-exclusive example.

Set of cell component data elements may be divided into the first cell component data elements 41A1 and second cell component data elements 41A2 for further analysis. In the illustrated example, first cell component data elements 41A1 include four single-channel data elements 20A1, 20A2, 20A3, 20A4, representing cell component types 21A1, 22A1, 23A1, 24A1 respectively. Second cell component data elements 41A2, in turn, include one single-channel data element 20A5, representing cell component type 25A.

It should be understood that, in some embodiments, different combinations of first and second cell component data elements 41A1 and 41A2 may be used, both in terms of number of cell component data elements picked as the first and the second (e.g., 4-to-1, 1-to-4, 3-to-2, 2-to-3 etc.), and in terms of cell component types included (e.g., first-cell component types 21A, 22A, 23A, 25A, second-cell component type 24A; or first-cell component types 21A, 22A, 24A, 25A, second-cell component type 23A etc.). Furthermore, in some embodiments, a plurality of said combinations may be used to assess presence of anomaly more reliably, as further described herein.

First cell component data elements 41A1 may then be provided as an input for ML-based model 51, which may be pretrained so as to obtain second cell component data element 41A2 in the reconstructed version 41A22, based on first cell component data element 41A1 in the original version 41A11. Hence, ML-based model 51 may be inferred on first cell component data element 41A1 in original version 41A11, to reconstruct second cell component data element 41A2 and obtain thereby second cell component data element 41A2 in reconstructed version 41A22.

Second cell component data element 41A2 in reconstructed version 41A22 may then be compared to second cell component data element 41A2 in original version 41A21. Next, a reconstruction error value may be calculated based on original version 41A21 and reconstructed version 41A22 (i.e., based on the comparison of the two versions). In case it is determined that the calculated reconstruction error value is higher than a predefined reconstruction error threshold value, the cell may be classified as having an anomaly.

The described concept provides technical improvement to known techniques since using one cell component data elements to reconstruct the other is inherently based on the assessment of cell inter-component (inter-organelle) organization.

In practice, with respect to specific cell-based research and phenotypic screening (e.g., identifying differences in cell populations with applications in identifying phenotypes signatures of specific diseases, drug screening, clustering compounds/genes into functional pathways, and identifying drugs targets/mechanisms of action etc.) it is essential to use both control cells and perturbed cells for the claimed concept.

More particularly, in some embodiments, the concept uses plurality of sets of cell component data elements in an original version (e.g., set 20A of single-channel data elements 20A1, 20A2, 20A3, 20A4 and 20A5), wherein each set corresponds to a distinct cell and each cell component data element within each set represents a distinct cell component type (e.g., cell component types 21A1, 22A1, 23A1, 24A1 and 25A1) of a respective cell. The plurality of sets of cell component data elements may include plurality of first sets, which may be sets corresponding to control cells of a cell-based research, and plurality of second sets, which may be sets corresponding to perturbed cells of the cell-based research. In order to achieve additional technical effect of increasing reliability and sensitivity of cell anomaly detection, ML-based model (e.g., ML-based model 51) should be respectfully trained based on sets corresponding to control cells. Then the ML-based model may be inferred on sets corresponding to perturbed cells, to assess whether each particular perturbation caused anomaly.

In particular, a training dataset for training ML-based model (e.g., ML-based model 51) may include examples of mapping between first cell component data elements (e.g., first cell component data element 41A1) of the first sets (i.e., sets corresponding to control cells) and second cell component data elements (e.g., second cell component data element 41A2) of the respective first sets. It should be understood that both the first and the second cell component data elements are represented in training dataset in respective original versions (e.g., original versions 41A11 and 41A21, respectively).

The ML-based model (e.g., ML-based model 51) may thus be trained, by using the training dataset, to reconstruct, based on the first cell component data elements (e.g., first cell component data elements 41A1) of first sets (i.e., sets corresponding to control cells) in the original version (e.g., original versions 41A11), second cell component data element (e.g., second cell component data element 41A2) of the first set in the original version (e.g., original versions 41A21), and obtain thereby the second cell component data element of the first set in a reconstructed version (e.g., reconstructed versions 41A22).

Then the reconstruction error threshold value should be set. In order to do that ML-based model (e.g., ML-based model 51) may then be inferred on first cell component data elements (e.g., first cell component data element 41A1) of the first sets (i.e., sets corresponding to control cells) in the original version (e.g., original versions 41A21), to obtain second cell component data elements of the first sets in the reconstructed version (e.g., reconstructed versions 41A22). Then a first reconstruction error values may be calculated based on the original versions (e.g., original versions 41A21) and the reconstructed versions (e.g., reconstructed versions 41A22) of second cell component data elements (e.g., second cell component data element 41A2) of the first sets. Then the reconstruction error threshold value may be defined based on the first reconstruction error values, e.g., based on the distribution of first reconstruction error values within the plurality of first sets (i.e., sets corresponding to control cells).

Respectively pretrained ML-based model (e.g., ML-based model 51) may then be inferred on first cell component data element (e.g., first cell component data element 41A1) of a second set (i.e., sets corresponding to perturbed cells) in the original version (e.g., original versions 41A21), to obtain second cell component data element of the second set in the reconstructed version (e.g., reconstructed versions 41A22).

Accordingly, since the ML-based model is pretrained on control cells and reconstruction error threshold value is set based on reconstruction made in respect to control cells, which do not have anomaly, the reconstruction error achieved in result of reconstruction of perturbed cells, which have anomaly, will deviate more distinctly, and thus anomaly will be more detectable.

In order to achieve additional technical effect by getting even higher sensitivity and reliability of anomaly detection, the following aspect should be considered. As known from the art, the lack of a straightforward meaning of key drivers of a network outcome is a widely perceived weakness of deep learning systems, which is especially critical in a clinical setting. Hence, if consider generative deep neural network (e.g., generative adversarial network (GAN)) as the basis for said ML-based model used in the described concept, training the ML-based model only on control cells will lead to more inevitable and detectable errors in reconstruction done with respect to perturbed cells, that have anomaly, since anomaly cells contain statistical properties that are different from control ones. Consequently, the aspects of deep learning neural networks that are commonly considered to be their disadvantages may be advantageously used in the claimed invention.

Hence, in some preferred embodiments of the claimed invention, the ML-based model is a generative deep neural network, e.g., generative adversarial network (GAN). In some embodiments, wherein microscopy images are used as cell component data elements, U-Net convolutional neural network may be used as the ML-based model. In alternative embodiments, wherein vector representations of sets of features extracted from microscopy images of cells are assessed, autoencoder neural network may be used as the ML-based model (e.g., ML-based model 51).

Reference is now made to FIG. 6, which depicts the system 10 for cell anomaly detection, according to some embodiments.

According to some embodiments of the invention, system 10 may be implemented as a software module, a hardware module, or any combination thereof. For example, system 10 may be or may include a computing device such as element 1 of FIG. 1. Furthermore, system 10 may be adapted to execute one or more modules of instruction code (e.g., element 5 of FIG. 1) to request, receive, analyze, calculate and produce various data. As further described in detail herein, system 10 may be adapted to execute one or more modules of instruction code (e.g., element 5 of FIG. 1) in order to receive a set of cell component data elements in an original version, wherein each cell component data element represents a distinct cell component type of a cell; infer at least one pretrained machine learning (ML)-based model on at least one first cell component data element of the set of cell component data elements in the original version, to obtain at least one second cell component data element of the set of cell component data elements in a reconstructed version; and classify the cell as having an anomaly 10A based on the reconstructed version of at least one second cell component data element etc.

As shown in FIG. 6, arrows may represent flow of one or more data elements to and from system 10 and/or among modules or elements of system 10. Some arrows have been omitted in FIG. 2 for the purpose of clarity.

In some embodiments, system 10 may include data input module 30 and data combination module 40, which may be performed as modules of instruction code (e.g., instruction code 5 of computing device 1, as shown in FIG. 4).

In some embodiments, data input module 30 may be configured to receive set of cell component data elements in an original version (e.g., set 20A of single-channel data elements), wherein each cell component data element (e.g., first-channel data element 21A, n-channel data element 22A) represents a distinct cell component type (e.g., cell component types 21A1, 22A1, 23A1, 24A1 and 25A1, shown in FIG. 5) of a cell (e.g., perturbed cell).

It should be understood, that depending on specific embodiment of the claimed invention, system 10 may be configured to analyze various number of cell component data elements (e.g., first-channel data element 21A, n-channel data element 22A) and combinations thereof per cell. Hence, in order to emphasize scalability of system 10, the number of respective elements is indicated as indefinite n.

In some embodiments, the set of cell component data elements (e.g., set 20A of single-channel data elements) comprises n distinct combinations of the at least one first and at least one second cell component data elements (e.g., first cell component data elements 41A1 and second cell component data element 41A2 shown in FIG. 5) of the set of cell component data elements (the division of the set of cell component data elements into “first” and “second” cell component data elements, as well as the purpose of such division is described in detail with reference to FIG. 5).

In some embodiments, data combination module 40 may be configured to receive cell component data elements (e.g., first-channel data element 21A, n-channel data element 22A) and form said n distinct combinations of the cell component data elements, wherein each combination includes first cell component data elements and second cell component data elements (same as first cell component data elements 41A1 and second cell component data elements 41A2, as shown in FIG. 5) in original versions 41A11 and 41A21, respectively.

In some embodiments, system 10 may comprise n ML-based models (e.g., first ML-based model 51, and n ML-based model 52). Each of n ML-based models is pretrained so as to obtain the at least one second cell component data element (e.g. second cell component data element 41A2 shown in FIG. 5) of a respective combination of the n distinct combinations in the reconstructed version (e.g., reconstructed version 41A22), based on the at least one first cell component data element (e.g. first cell component data elements 41A1 shown in FIG. 5) of the respective combination of the n distinct combinations in the original version (e.g., original version 41A11, as shown in FIG. 5).

Accordingly, first ML-based model 51 may be configured to receive first input combination 41A1 in original version 41A11 (same as first cell component data elements 41A1 shown in FIG. 5), and n ML-based model 52 may be configured to receive n input combination 42A1 in original version 42A11, accordingly.

Accordingly, first ML-based model 51 may be configured to output first-channel data element 41A2 (same as second cell component data element 41A2 shown in FIG. 5) in reconstructed version 41A22, and n ML-based model 52 may be configured to output n-channel data element 42A2 in reconstructed version 42A22, respectively.

In some embodiments, system 10 may include data comparison module 60, which may be performed as module of instruction code (e.g., instruction code 5 of computing device 1, as shown in FIG. 4).

Data comparison module 60 may be configured to receive second cell component data elements (same as second cell component data elements 41A2, as shown in FIG. 5) of each of n distinct combinations in their original versions (e.g., original version 41A21, as shown in FIG. 5) from data combination module 40. Data comparison module 60 may be further configured to receive first-channel data element 41A2 (same as second cell component data element 41A2 shown in FIG. 5) in reconstructed version 41A22, and n-channel data element 42A2 in reconstructed version 42A22, respectively. Data comparison module 60 may be further configured to receive reconstruction error threshold setup 60A, which may include predetermined reconstruction error threshold values for each of n combinations. It should be understood that each of n combinations may have either equal or different reconstruction error threshold values.

Data comparison module 60 may be further configured to compare, for each of n combinations, second cell component data elements in original version with second cell component data elements in reconstructed version (same as second cell component data elements 41A2 in original version 41A21 with second cell component data elements 41A2 in reconstructed version 41A22, as shown in FIG. 5).

Data comparison module 60 may be further configured to calculate, for each combination of the n distinct combinations, a reconstruction error value based on the original version and the reconstructed version of respective second cell component data element, i.e., based on said comparison. Data comparison module 60 may be further configured to compare calculated reconstruction error values with respective reconstruction error threshold values of reconstruction error threshold setup 60A.

Data comparison module 60 may be further configured to classify the cell (e.g., perturbed cell) as having an anomaly 10A by determining that at least one of the calculated reconstruction error values is higher than a respective predefined reconstruction error threshold value. Data comparison module 60 may be further configured to output respective anomaly detection result 10A, indicating whether the cell has anomaly or not.

It should be understood that the claimed invention is not limited to the abovementioned logic of considering plurality of calculated reconstruction error values in order to perform classification. For instance, value of each combination of n distinct combinations and each ML-based model of n ML-based models may be adjusted by using different weight coefficients assigned to respective results of comparison of reconstruction error values with respective thresholds. This way the fact that some cell component data elements (and some cell components, respectively) may be reconstructed easier (i.e., with less reconstruction error value) than the others, may be considered and the respective results may be equalized.

It should be understood that provided examples of logic of considering plurality of calculated reconstruction error values in order to perform classification are non-exclusive and any other logic is also covered by the essence of the claimed invention.

In some embodiments, system 10 may include a training module 53 configured to perform training of ML-based models (e.g., first ML-based model 51 and n ML-based model 52), as well as setting reconstruction error threshold values and recording them as reconstruction error threshold setup 60A, as described with respect to FIG. 5.

As known in the art, the term “phenotypic screening” may be used herein to refer to any type of screening that may be used in biological research, diagnostic procedures and drug discovery, to identify substances such as small molecules, peptides, RNA molecules, and the like, which may alter a phenotype of a cell or an organism in a specific manner.

As explained herein, cell anomaly 10A may include, represent or may be characteristic of a specific, anomalous phenotypic signature. Such anomalous phenotypic signature may in turn, be characteristic of, or represent a specific condition, such as a disease of the cell, a mechanism of a biochemical, functional pathway that is operating in the cell, a response to a specific drug or treatment administered to the cell, and the like. Therefore, system 10 may utilize classified anomaly 10A for various aspects of phenotypic screening.

For example, system 10 may include (e.g., as depicted in FIG. 6), or may be communicatively connected to (e.g., via a computer communication networks) a database of cellular phenotypic information 61. Data comparison module 60 may transmit anomaly 10A to phenotype database 61, and may retrieve (e.g., as a response) one or more recommendation data elements 10B pertaining to a condition of the depicted cell. In other words, cell-anomaly classification results 10A may be utilized by system 10 to produce a variety of recommendations and/or notifications 10B that pertain to phenotypic screening of the relevant (e.g., depicted) cell(s).

For example, recommendations 10B, may include a suggested diagnosis of the depicted cell(s) or an organism of the cell (e.g., from which the cell was extracted). In another example, recommendations 10B, may include a recommendation for treatment, such as a selection of a drug and/or dosage thereof, to be administered to the depicted cell and/or organism. In another example, recommendations 10B may include an indication of a biochemical pathway that is active within the cell(s), e.g., in association with said treatment or drug. In yet another example, recommendations 10B may include an indication of abundance of one or more molecules (e.g., RNA molecules, proteins, and the like) in the relevant cell(s). Other such examples of recommendations or indications 10B of phenotypic screening may also be possible.

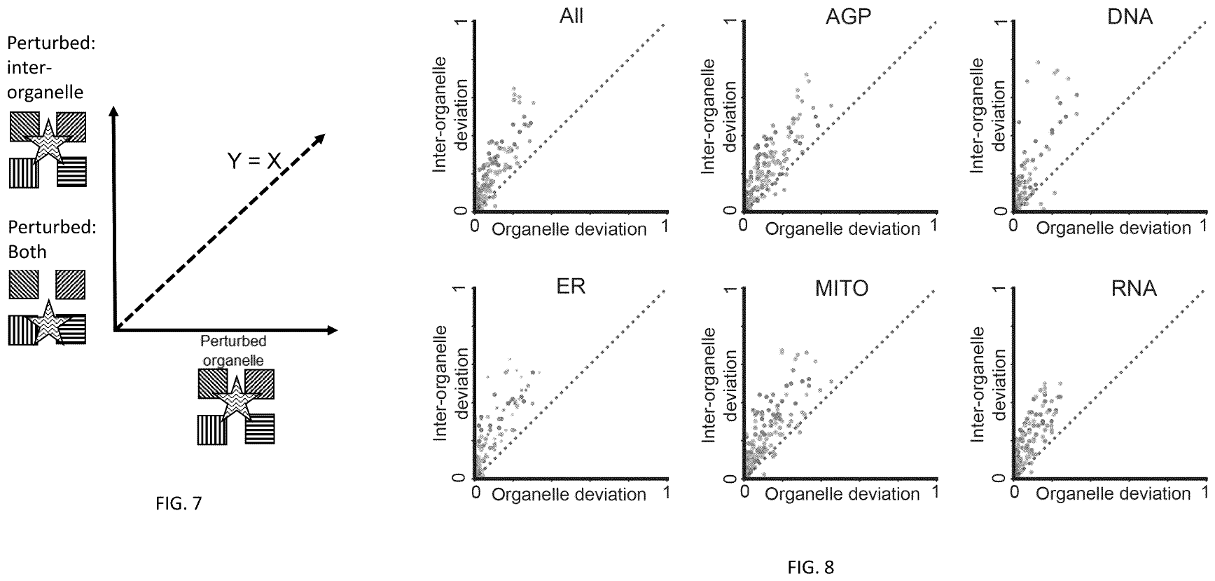

Reference is now made to FIG. 7, which is a plot, depicting evaluation of the magnitude of perturbed inter-organelle organization vs. the corresponding organelle properties.

The illustrated plot represents the approach of evaluation of the magnitude of perturbed inter-organelle organization vs. the corresponding organelle properties. Each observation will indicate the normalized deviation of organelle properties from the control (X-axis) and the corresponding normalized mapping reconstruction error (Y-axis). It should be understood that values above the diagonal will indicate enhanced sensitivity of the claimed readout.

During practical approbation of the claimed invention, the sensitivity of the proposed readout was assessed and compared to the current standard readouts (referring to FIG. 2, cases like shown in upper right corner vs. bottom right corner). The abundance of “broken” relations was evaluated-channels (e.g., single-channel data elements 20A1, 20A2, 20A3, 20A4 and 20A5) that do not deviate much from the control (e.g., from control cells that formed the basis for training ML-based models, like first ML-based model 51, and n ML-based model 52, shown in FIG. 6) in terms of their feature representation alone, but their predicted mapping from the other four channels does (referring to FIG. 2, cases like shown in bottom-left corner). To assess “mechanistic interpretability” the alterations of all five ML-based models were measured, each mapping four-to-one fluorescent channel to test the hypothesis that some perturbations “break” some, but not other, inter-organelle spatial dependencies.

Reference is now made to FIG. 8, which is a set of plots, depicting normalized deviation in the corresponding organelle properties in relation to the deviation in the inter-organelle organization.

Each plot depicts the normalized (in respect to the controls (control cells)) deviation in the corresponding organelle properties (X-axis) in relation to the deviation in the inter-organelle organization (Y-axis), which were achieved during practical approbation of the claimed invention. Each data point is the mean normalized value for all cells in a given well. Color indicates plate. Remark “All” means that the plot shows the combined effect for all features across the five channels (e.g., single-channel data elements 20A1, 20A2, 20A3, 20A4 and 20A5, for which AGP, DNA, ER, MITO, RNA were used respectively). The feature cutoff is Z-score ≥15 (results were consistent to other cutoffs—data not shown). As can be seen, vast majority of “hits” are above the Y=X diagonal, establishing inter-organelle organization as a sensitive readout for image-based cell phenotyping.

To evaluate the potential of the claimed approach the approbation was focused on a subset of 35 plates (11197 wells) from 27 (the full dataset includes 400 plates). Every single cell was represented with a feature vector that included five sets of features, extracted from each of the fluorescent channels. All features were extracted and included in the dataset. Then five generative deep neural network autoencoders were trained based on control cells, each network (such as first ML-based model 51, and n ML-based model 52, shown in FIG. 6) was trained to map a different combination of 4-to-1 feature sets (derived from the different fluorescent image channels, same as combinations of cell component data elements formed by data combination module 40, as described with reference to FIG. 6). Then the statistics of the reconstruction error in control cells was assessed and used to define a normalized measure of deviation of a perturbed well from the variation of the control wells (i.e., used to define a reconstruction error threshold value, as described with reference to FIGS. 5 and 6). It is essential that different plates for training and testing were used to avoid bias due to plate-plate variability. To evaluate the sensitivity of the claimed readout, direct per-well comparison was performed, the comparison between the state-of-the-art readout of the normalized organelle deviation from the control wells variability and the normalized deviation in the organelle reconstruction error (i.e., reconstruction error value) in respect to the variability of reconstruction error value in control wells.

Values above the Y=X diagonal indicate perturbed wells where the inter-organelle organization deviate from the control's inter-organelle organization more than the deviation of the corresponding organelle properties. The demonstrated results are based on measurement of the fraction of features above a given threshold (results were validated for a range of thresholds-data not shown).

Preliminary results establish that inter-organelle organization is a sensitive readout, as indicated by the vast majority of hits deviating (from the control wells) more in their inter-organelle organization in relation to deviation in the organelle properties.

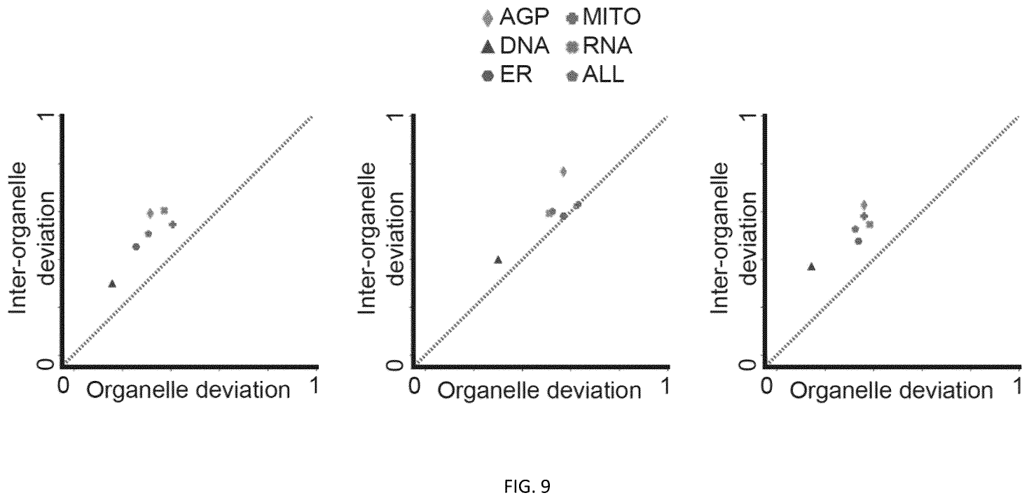

Reference is now made to FIG. 9, which is a set of plots, depicting replication of anomaly detection results 10A across different plates during approbation of the claimed invention.

As shown by replicating phenotype across three different plates, deviations in DNA properties and reconstruction error values were smaller than in the other channels. Shown feature cutoff of Z-score ≥6.

Reproducibility of the readout was verified by consistent deviation of the different organelles in response to a common “hit” perturbation across plates as verified by subjective visual assessment.

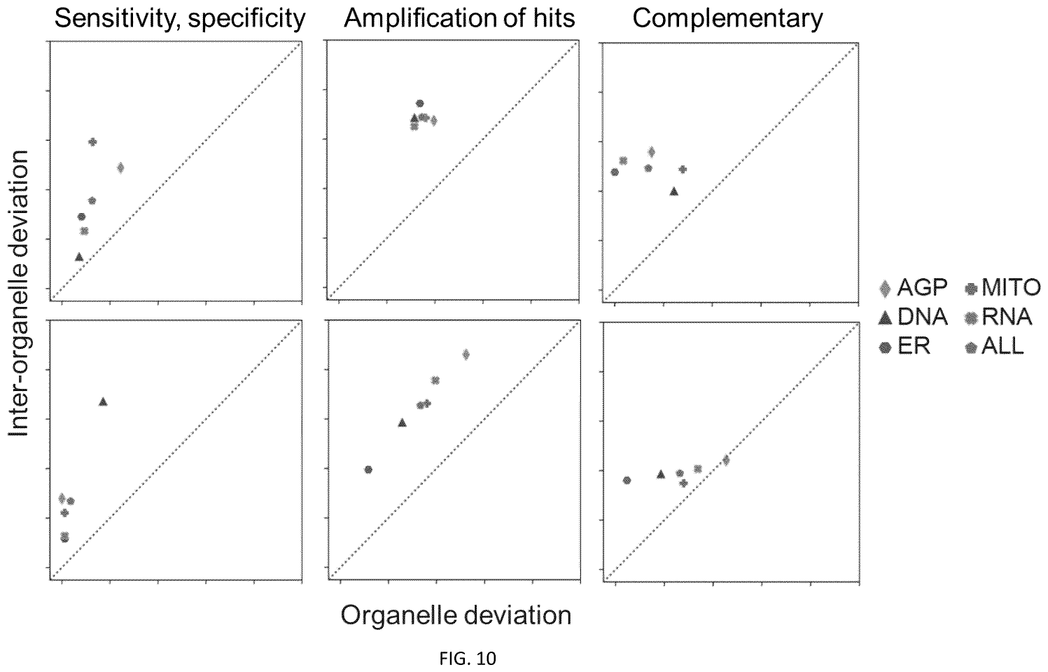

Reference is now made to FIG. 10, which is a set of plots, depicting sensitivity and specificity of inter-organelle organization approach in relation to the single organelle approach.

As can be seen from the provided plots, inter-organelle deviation is a complementary readout that is more sensitive and specific. Left: spatial inter-organelle dependencies provide more specific and interpretable readout. Middle: obvious hits are amplified in their disruption. Right: inter-organelle organization is a complementary readout to organelle properties. Shown feature cutoff of Z-score ≥6.

Preliminary results also suggest that: (1) hits in organelle properties are amplified in the inter-organelle organization-subtle phenotypes are amplified making it easier to identify significant phenotypes (middle column represented in FIG. 10); (2) inter-organelle organization phenotypes that are missed by traditional analyses can be discovered (FIG. 10, left column); (3) spatial inter-organelle dependencies are differentially determined based on organelle composition and perturbation, implying a more specific and interpretable readout: different phenotype magnitudes to different organelles (FIG. 10, left column); (4) inter-organelle organization is a complementary readout to organelle properties: the same deviation across organelles in inter-organelle organization/organelle properties can be mapped to different deviation in the corresponding organelle properties/inter-organelle organization (FIG. 10, left versus right column).

The complete validation of the methodology may be provided by the following steps: (1) assessing reproducibility by verifying that hits are replicated (see preliminary results); (2) evaluating similarities along signaling pathways; (3) evaluating the dose-dependent response; (4) replicating the validation and predicting phenotypic similarity based on gene ontologies annotations.

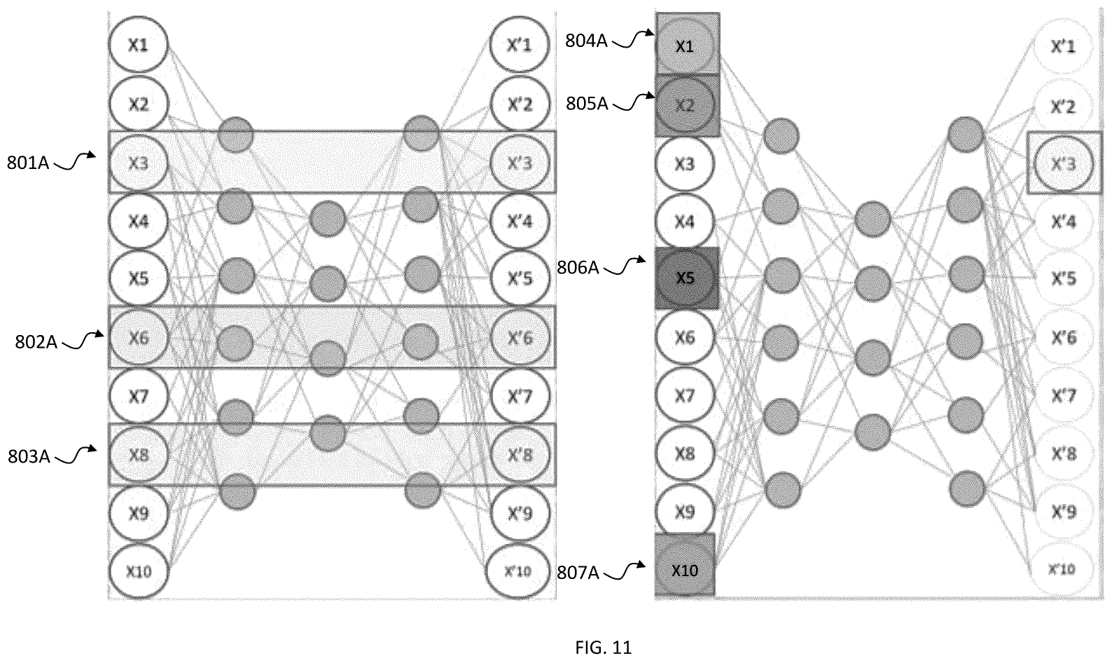

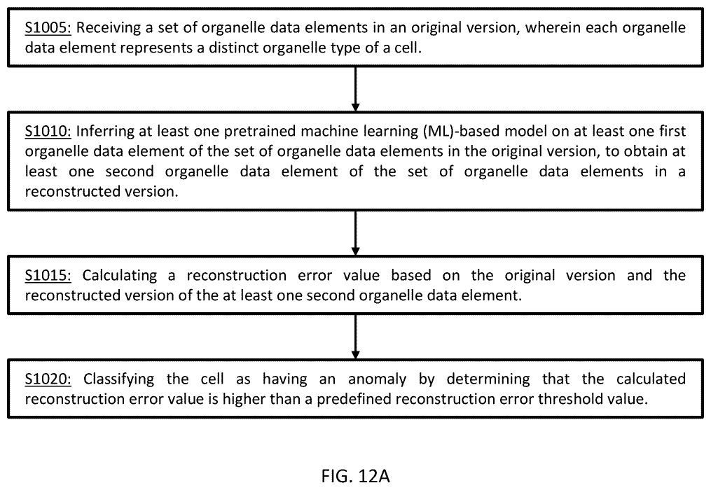

Reference is now made to FIG. 11, which is a schematic representation, depicting anomaly detection for model (e.g., first ML-based model 51) explainability.

As can be seen from the figure, the left part represents schematic representation of an autoencoder (e.g., first ML-based model 51) that fail to reconstruct the anomalous features 801A, 802A and 803A. The right part shows the interpretation of that the anomaly 10A in 801A is explained by the combined alteration of features 804A, 805A, 806A and 807A.

For practical approbation, the systematic comparison between neural-network-based image-derived and engineered feature representations was made to quantitatively characterize the benefits and phenotype scoring capacities of manually engineered features of single cells versus automatically deep-learned extracted features of image patches (without single cell resolution). For the engineered single cell feature representations, autoencoder networks (e.g., first ML-based model 51, and n ML-based model 52, shown in FIG. 6) were trained while for the image representation the U-Net convolutional neural network was used. This comparison was made to establish which are the better readouts in term of sensitivity.

The lack of a straightforward meaning of key drivers of a network outcome is a widely perceived weakness of deep learning systems, which is especially critical in a clinical setting. Consequently, the developed method takes advantage of the asymmetry between the large numbers of controls in comparison to the limited number of replicates at each perturbed conditions by applying the claimed anomaly detection method to identify how perturbed cells deviate from the control. A generative adversarial network (GAN) was trained using control cells, since it may easily fail to reconstruct representations of perturbed cells that are associated with different statistical properties. This allows to identify perturbed cells as anomalous in respect to the expected representation based on the statistics of the control cells. To pin-point what combination of features (or combination of cell component data elements) define an instance as anomalous, an unsupervised extension may be used of a game theory-based method for interpreting models' predictions. In some embodiments, the method may assign each cell feature an importance value for each particular reconstruction to provide different quantitative explanation for different forms of anomalies induced by different perturbations.

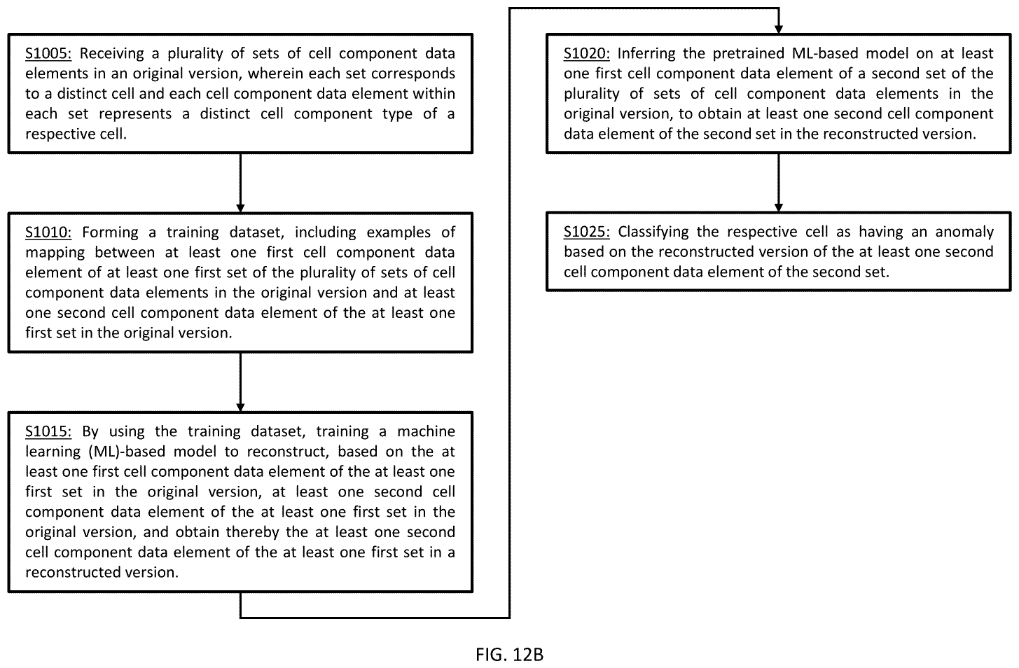

Referring now to FIG. 12A, a flow diagram is presented, depicting a method of cell anomaly 10A detection, by at least one processor, according to some embodiments.

As shown in step S1005, the at least one processor (e.g., processor 2 of FIG. 4) may perform receiving a set of cell component data elements (e.g., set 20A of single-channel data elements 20A1, 20A2, 20A3, 20A4 and 20A5) in an original version (e.g., original versions 41A11, 41A21 and 42A11) wherein each cell component data element (e.g., single-channel data elements 20A1, 20A2, 20A3, 20A4 and 20A5) represents a distinct cell component type (e.g., cell component types 21A1, 22A1, 23A1, 24A1 and 25A1) of a cell. Step S1005 may be carried out by data input module 30 (as described with reference to FIG. 6).

As shown in step S1010, the at least one processor (e.g., processor 2 of FIG. 4) may perform inferring of at least one pretrained machine learning (ML)-based model (e.g., first ML-based model 51 and n ML-based model 52) on at least one first cell component data element (e.g., first cell component data elements 41A1) of the set of cell component data elements (e.g., set 20A of single-channel data elements 20A1, 20A2, 20A3, 20A4 and 20A5) in the original version (e.g., original version 41A11), to obtain at least one second cell component data element (e.g., second cell component data element 41A2) of the set of cell component data elements in a reconstructed version (e.g., reconstructed version 41A22). Step S1010 may be carried out by first ML-based model 51 and n ML-based model 52 (as described with reference to FIG. 6).

As shown in step S1015, the at least one processor (e.g., processor 2 of FIG. 4) may classify the cell as having an anomaly 10A based on the reconstructed version (e.g., reconstructed version 41A22) of at least one second cell component data element (e.g., second cell component data element 41A2). Step S1015 may be carried out by data comparison module 60 (as described with reference to FIG. 6).

Referring now to FIG. 12B, a flow diagram is presented, depicting a method of cell anomaly 10A detection, by at least one processor, according to some alternative embodiments.

As shown in step S1005, the at least one processor (e.g., processor 2 of FIG. 4) may perform receiving a plurality of sets of cell component data elements (e.g., set 20A of single-channel data elements 20A1, 20A2, 20A3, 20A4 and 20A5) in an original version (e.g., original versions 41A11, 41A21 and 42A11), wherein each set corresponds to a distinct cell and each cell component data element (e.g., single-channel data elements 20A1, 20A2, 20A3, 20A4 and 20A5) within each set represents a distinct cell component type (e.g., cell component types 21A1, 22A1, 23A1, 24A1 and 25A1) of a respective cell. Step S1005 may be carried out by data input module 30 (as described with reference to FIG. 6).

As shown in step S1010, the at least one processor (e.g., processor 2 of FIG. 4) may perform forming a training dataset, including examples of mapping between at least one first cell component data element (e.g., first cell component data elements 41A1) of at least one first set of the plurality of sets of cell component data elements (e.g., set 20A of single-channel data elements 20A1, 20A2, 20A3, 20A4 and 20A5) in the original version (e.g., original version 41A11) and at least one second cell component data element (e.g., second cell component data element 41A2) of the at least one first set in the original version (e.g., original version 41A21). Step S1010 may be carried out by system 10, e.g., by training module (as described with reference to FIG. 6).

As shown in step S1015, the at least one processor (e.g., processor 2 of FIG. 4) may perform training, by using the training dataset, of at least one machine learning (ML)-based model (e.g., first ML-based model 51 and n ML-based model 52) to reconstruct, based on the at least one first cell component data element (e.g., first cell component data elements 41A1) of the at least one first set (e.g., set 20A) in the original version (e.g., original version 41A11), at least one second cell component data element (e.g., second cell component data element 41A2) of the at least one first set (e.g., set 20A) in the original version (e.g., original version 41A21), and obtain thereby the at least one second cell component data element (e.g., second cell component data element 41A2) of the at least one first set in a reconstructed version (e.g., reconstructed version 41A22). Step S1015 may be carried out by system 10, e.g., by training module (as described with reference to FIG. 6).

As shown in step S1020, the at least one processor (e.g., processor 2 of FIG. 4) may perform inferring the pretrained at least one ML-based model (e.g., first ML-based model 51 and n ML-based model 52) on at least one first cell component data element (e.g., first cell component data elements 41A1) of a second set (e.g., set 20A) of the plurality of sets of cell component data elements in the original version (e.g., original version 41A21), to obtain at least one second cell component data element (e.g., second cell component data element 41A2) of the second set in the reconstructed version (e.g., reconstructed version 41A22). Step S1020 may be carried out by first ML-based model 51 and n ML-based model 52 (as described with reference to FIG. 6).

As shown in step S1025, the at least one processor (e.g., processor 2 of FIG. 4) may classify the respective cell as having an anomaly 10A based on the reconstructed version (e.g., reconstructed version 41A22) of the at least one second cell component data element (e.g., second cell component data element 41A2) of the second set (e.g., set 20A). Step S1025 may be carried out by data comparison module 60 (as described with reference to FIG. 6).

Claimed method provides a sensitive and specific interpretable functional readouts, complementary to the current measurements in high-content image-based phenotyping, with broad translational applicability in drug discovery, repurposing existing drugs, and lead-hopping. One immediate potential translational impact is the ability to identify phenotyping alterations that are missed by current existing readouts. In other words, in cases with a drug that, in addition to transforming cells to their “healthy” state, also perturb the internal sub-cellular organization, the claimed methodology could identify this defect and avoid expensive follow-up validations. Another potential translational impact is in combinatorial drug therapy, i.e., prediction of combinations and dosages of FDA-approved drugs that will synergistically provide precise and effective treatment, for targeting defects in organelle-organelle interactions by seeking orthogonal combinations of drugs where each drug “fixes” a different “broken” inter-organelle relation. For example, given the current failure in finding a single drug with a strong healing potential (and minimal side effects) for COVID-19, proposed approach may assist in the prediction of combinations and dosages of FDA-approved drugs that will synergistically provide precise and effective COVID-19 treatment.

As can be seen, the new methodology to measure alterations in inter-organelle spatial dependencies was developed. It was applied to identify new treatments that interfere with specific spatial dependencies between organelles by analyzing publicly available high-content imaging-based screening data.

Hence, current invention is a new image-based high-content phenotyping readout for specific interferences in organelle-organelle spatial organization, and the identification of new putative drugs hypothesized to alter inter-organelle organization. Preliminary results indicate that not only the disruption of inter-organelle spatial organization is a more sensitive readout, but it can also identify phenotypes that are completely missed in the traditional analysis of pooling image-based features across all fluorescent channels. Altogether, the contribution includes: the first systematic quantitative readout for inter-organelle organization phenotyping; a more sensitive readout that is also complementary to the current state of the art; phenotype amplification making it easier to identify subtle phenotypes; discovery of new phenotypes that are missed by traditional analyses; a more specific and interpretable readout differentially determined for each set of inter-organelle spatial dependencies.

While current computational approaches pool image-based features from different modalities, each of a distinct organelle, the proposed one provides new methodology to measure alterations in the spatial dependencies between different organelles, and apply it to identify new treatments that interfere with specific spatial dependencies between organelles. This will enable discovery and mechanistic interpretability of the effects each treatment has on specific aspects of cell organization in terms of “breaking” existing relations between multiple cell structures, which are currently inaccessible.

As elaborated herein, preliminary results indicate that inter-organelle organization are complementary, sensitive and specific readouts for high-content phenotyping applications. These results along with the approbation by reliance of the project on existing public dataset, prior experience in bioimage data reuse and in similar computational techniques, which provides a competitive edge and reduce the risks of problems of practical implementation.

The claimed invention was practically approbated on three large-scale and high-quality publicly available cell-painting datasets. The first is a small molecule compound screen, that includes both the raw images as well as engineered features extracted from these images. The second is an overexpression screen, with information regarding specific pathways that can be explored and used for further validations. The third is a COVID-19 drug screen of normal human kidney cells treated with drugs and compounds in a dose-dependent manner.

Therefore, the proposed technology may be the first to target defects in inter-organelle spatial dependencies and holds the promise for broad translational applicability in drug discovery, repurposing existing drugs, and combinatorial drug therapy.

As can be seen from the provided description, the claimed invention represents the system and method of cell anomaly detection 10A, which increases reliability of cell anomaly detection. More specifically, the claimed invention provides an assessment of cell inter-component (inter-organelle) organization for detecting cell anomaly 10A.

As described, embodiments of the invention may include a practical application for method and system of cell anomaly detection 10A in different fields of endeavor. Embodiments of the claimed invention may thus provide an improvement in the technological field of computer assisted cell anomaly diagnostics.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of cell anomaly detection by at least one processor, the method comprising:
receiving a set of cell component data elements in an original version, wherein each cell component data element represents a distinct cell component type of a cell;
inferring at least one pretrained machine learning (ML)-based model on at least one first cell component data element of the set of cell component data elements in the original version, to obtain at least one second cell component data element of the set of cell component data elements in a reconstructed version;
classifying the cell as having an anomaly based on the reconstructed version of at least one second cell component data element.

2. The method according to claim 1, further comprising:
communicating the classification of anomaly to a database of cellular phenotypic information; and
obtaining, from the database, one or more recommendation data elements pertaining to a condition of the cell, said recommendation data elements selected from a list consisting of: a suggested diagnosis of an organism of the cell, a recommendation for drug treatment of the organism, a recommendation for drug dosage, to be administered to the organism, and an indication of a biochemical pathway that is associated with said treatment.

3. The method according to claim 1, wherein classifying the cell as having an anomaly comprises
calculating a reconstruction error value based on the original version and the reconstructed version of the at least one second cell component data element;
classifying the cell as having an anomaly, further based on the calculated reconstruction error value.

4. The method according to claim 1, wherein classifying the cell as having an anomaly based on the calculated reconstruction error value further comprises classifying the cell as having an anomaly by determining that the calculated reconstruction error value is higher than a predefined reconstruction error threshold value.

5. The method according to claim 1, wherein the at least one pretrained ML-based model is pretrained so as to obtain the at least one second cell component data element in the reconstructed version, based on the at least one first cell component data element in the original version.

6. The method according to claim 1, wherein the at least one cell component data element is a microscopy image of the cell.

7. The method according to claim 1, wherein the at least one original data element is a vector representation of a set of features extracted from a microscopy image of the cell.

8. The method according to claim 1, wherein the set of cell component data elements comprises n distinct combinations of the at least one first and at least one second cell component data elements,
and wherein the at least one ML-based model comprises n ML-based models, each corresponding to a respective combination of the n distinct combinations.

9. The method according to claim 8, wherein each ML-based model of the n ML-based models is pretrained to obtain the at least one second cell component data element in the reconstructed version, based on the at least one first cell component data element in the original version, according to the respective combination of the n distinct combinations.

10. The method according to claim 1, wherein inferring the at least one pretrained machine learning (ML)-based model comprises
inferring each ML-based model of the n ML-based models on the at least one first cell component data element of the respective combination in the original version, to obtain the at least one second cell component data element of the respective combination in the reconstructed version.

11. The method according to claim 8, wherein classifying the cell as having an anomaly comprises:
for each combination of the n distinct combinations, calculating a reconstruction error value based on the original version and the reconstructed version of at least one respective second cell component data element;
classifying the cell as having an anomaly, further based on the calculated reconstruction error values.

12. The method according to claim 1, wherein classifying the cell as having an anomaly based on the calculated reconstruction error values further comprises classifying the cell as having an anomaly by determining that at least one of the calculated reconstruction error values is higher than a respective predefined reconstruction error threshold value.

13. A method of cell anomaly detection by at least one processor, the method comprising:
receiving a plurality of sets of cell component data elements in an original version, wherein each set corresponds to a distinct cell and each cell component data element within each set represents a distinct cell component type of a respective cell;
forming a training dataset, including examples of mapping between at least one first cell component data element of at least one first set of the plurality of sets of cell component data elements in the original version and at least one second cell component data element of the at least one first set in the original version;
by using the training dataset, training at least one machine learning (ML)-based model to reconstruct, based on the at least one first cell component data element of the at least one first set in the original version, at least one second cell component data element of the at least one first set in the original version, and obtain thereby the at least one second cell component data element of the at least one first set in a reconstructed version;
inferring the pretrained at least one ML-based model on at least one first cell component data element of a second set of the plurality of sets of cell component data elements in the original version, to obtain at least one second cell component data element of the second set in the reconstructed version; and
classifying the respective cell as having an anomaly based on the reconstructed version of the at least one second cell component data element of the second set.

14. The method of claim 13, wherein the method further comprises
calculating a first reconstruction error value based on the original version and the reconstructed version of the at least one second cell component data element of the at least one first set;
defining a reconstruction error threshold value based on the first reconstruction error value.

15. The method of claim 14, wherein the at least one first set comprises a plurality of the first sets, and wherein the method further comprises defining the reconstruction error threshold value based on a distribution of first reconstruction error values within the plurality of first sets.

16. The method of claim 14, wherein classifying the respective cell as having an anomaly comprises
calculating a second reconstruction error value based on the original version and the reconstructed version of the at least one second cell component data element of the at least one second set;
classifying the respective cell as having an anomaly by determining that the second reconstruction error value is higher than the predefined reconstruction error threshold value.

17. The method of claim 13, wherein the at least one first set of the plurality of sets of cell component data elements in the original version corresponds to a distinct control cell of a cell-based research and the at least one second set of the plurality of sets of cell component data elements in the original version corresponds to a distinct perturbed cell of the cell-based research.

18. The method of claim 13, wherein the at least one ML-based model is a generative deep neural network.

19.-20. (canceled)

21. A system for cell anomaly detection, the system comprising: a non-transitory memory device, wherein modules of instruction code are stored, and at least one processor associated with the memory device, and configured to execute the modules of instruction code, whereupon execution of said modules of instruction code, the at least one processor is configured to:
receive a set of cell component data elements in an original version, wherein each cell component data element represents a distinct cell component type of a cell;
infer at least one pretrained machine learning (ML)-based model on at least one first cell component data element of the set of cell component data elements in the original version, to obtain at least one second cell component data element of the set of cell component data elements in a reconstructed version;
classify the cell as having an anomaly based on the reconstructed version of at least one second cell component data element.

22. The system of claim 21, wherein the at least one processor is further configured to:
calculate a reconstruction error value based on the original version and the reconstructed version of the at least one second cell component data element; and
classify the cell as having an anomaly, further based on the calculated reconstruction error value.

23.-31. (canceled)
